(19) United States
(12) Patent Application Publication
(10) Pub. No.: US 2013/0280745 A1
(43) Pub. Date: Oct. 24, 2013

(54) HIGH-THROUGHPUT, SENSITIVE DETECTION OF GLUCOSE-6-PHOSPHATE DEHYDROGENASE

(71) Applicant: Bruno Casetta, Sacile (IT)

(72) Inventor: Bruno Casetta, Sacile (IT)

(73) Assignee: DH Technologies Development Pte. Ltd., Singapore (SG)

(21) Appl. No.: 13/989,323

(22) PCT Filed: Nov. 17, 2011
(86) PCT No.: PCT/US11/61207

§ 371 Date: Jun. 26, 2013

Related U.S. Application Data
(60) Provisional application No. 61/416,951, filed on Nov. 24, 2010.

(51) Int. Cl. C12Q 1/32

(52) U.S. Cl. CPC C12Q 1/32
USPC 435/26

(57) ABSTRACT

Provided herein are methods for determining an amount of glucose-6-phosphate dehydrogenase (“G6PDH”) in a biological sample. Also provided herein are methods for detecting G6PDH deficiency, as well as diagnosing G6PDH-associated disorders, such as acute hemolytic anemia and pre-eclampsia.

### RELATED APPLICATIONS

This application is related and claims priority to U.S. Provisional Application Ser. No. 61/416,957, filed Nov. 24, 2010. The entire contents of this application are expressly incorporated herein by this reference.

### BACKGROUND

Glucose-6-phosphate dehydrogenase (“G6PD” or “G6PDH”) is a cytosolic enzyme in the pentose phosphate pathway (also referred to as the phosphogluconate pathway). This metabolic pathway generates pentoses and nicotinamide adenine dinucleotide phosphate (“NADPH”) in cells, such as erythrocytes, thus supplying reducing energy. NADPH also maintains the level of glutathione in cells, which helps protect red blood cells against oxidative damage. Glucose-6-phosphate dehydrogenase is stimulated by its substrate, Glucose-6-phosphate (“G6P”), to form 6-phosphoglucono-δ-lactone. It is the rate-limiting enzyme of the pentose phosphate pathway.

Genetic deficiency of G6PDH in humans predisposes them to certain disorders, such as non-immune hemolytic anemia. Present methods for determining the presence of G6PDH and/or G6PDH activity include qualitative or quantitative fluorescent screening. However, such methods are often subjective and are generally incapable of detecting a partial deficiency of G6PDH in many cases. See, e.g., Reclos G J, et al. “Glucose-6-Phosphate Dehydrogenase Deficiency Neonatal Screening” J Med Screen, 7: 46-51 (2000) and Kaplan M, et al. “Comparison of Commercial Screening Tests for Glucose-6-Phosphate Dehydrogenase Deficiency in the Neonatal Period” Clin Chem, 43(7): 1236-37 (1997).

### SUMMARY

Accordingly, in some embodiments, applicant's teachings provide high throughput methods for detecting an amount of G6PDH in a biological sample. The methods include, for example: reacting a biological sample with G6P and NADP in the presence of a surfactant and a buffer under suitable conditions to form 6-phosphogluconic acid; quenching the biological sample to form a quenched sample; and detecting by mass spectrometry a presence or amount of 6-phosphogluconic acid in the quenched sample, wherein the amount of 6-phosphogluconic acid in the quenched sample is related to the amount of G6PDH in the biological sample.

In some embodiments, the quenching is accomplished using an enzyme denaturant.

In some embodiments, the mass spectrometry is tandem mass spectrometry. In some embodiments, the mass spectrometry is accomplished using a mass spectrometer equipped with a thermally assisted electrospray ionization probe, e.g., a TurboIonSpray® (TIS) probe. In some embodiments, the mass spectrometry is accomplished using a mass spectrometer equipped with a reversed phase liquid chromatography column.

In some embodiments, the detecting occurs in less than about 5 minutes. In some embodiments, the detecting occurs in less than about 3 minutes. In some embodiments, the method allows for detection of about 0.01 milliUnits of G6PDH or less. In some embodiments, the detecting is not substantially affected by fluctuations in temperature.

In some embodiments, applicant's teachings provide methods for detecting G6PDH deficiency in a subject. The methods include, for example: determining by mass spectrometry an amount of G6PDH in a biological sample from a subject; and comparing the amount of G6PDH in the biological sample to an appropriate control, wherein G6PDH deficiency is detected when the amount of G6PDH in the sample is less than the appropriate control.

In some embodiments, the subject is a neonate or an infant. In some embodiments, the subject is a pregnant woman.

In some embodiments, an amount of G6PDH in the sample which is 10% less than the control indicates a G6PDH deficiency in the subject. In some embodiments, an amount of G6PDH in the sample which is 25% less than the control indicates a G6PDH deficiency in the subject. In some embodiments, an amount of G6PDH in the sample which is 50% less than the control indicates a G6PDH deficiency in the subject.

In some embodiments, detecting G6PDH deficiency further includes: reacting the biological sample with G6P and NADP in the presence of a surfactant and a buffer under suitable conditions to form 6-phosphogluconic acid; quenching the biological sample to form a quenched sample; and detecting by mass spectrometry a presence or amount of 6-phosphogluconic acid in the quenched sample, wherein the amount of 6-phosphogluconic acid in the quenched sample is related to the amount of G6PDH in the biological sample.

In some embodiments, applicant's teachings provide methods for diagnosing acute hemolytic anemia in a subject. The methods include, for example: detecting G6PDH deficiency in a subject in accordance with applicant's teachings, wherein a G6PDH deficiency in the subject indicates acute hemolytic anemia in the subject. In some embodiments, an amount of G6PDH in the sample which is 25% less than the control indicates acute hemolytic anemia in the subject.

In some embodiments, applicant's teachings provide methods for diagnosing pre-eclampsia in a subject. The methods include, for example: detecting G6PDH deficiency in a subject in accordance with applicant's teachings, wherein a G6PDH deficiency in the subject indicates pre-eclampsia in the subject. In some embodiments, an amount of G6PDH in the sample which is 25% less than the control indicates pre-eclampsia in the subject.

In some embodiments, applicant's teachings provide prognostic methods for increased mortality and/or morbidity resulting from pre-eclampsia or acute hemolytic anemia in a subject. The methods include, for example: detecting G6PDH deficiency in a subject in accordance with applicant's teachings, wherein an amount of the G6PDH in the biological sample which is 50% or less than the control indicates a prognosis of increased mortality and/or morbidity in the subject.

In some embodiments, the appropriate control is a control based upon G6PDH levels in a normal population. In some embodiments, the biological sample is a dried blood sample.

In some embodiments, applicant's teachings provide kits for detecting an amount of G6PDH in a biological sample. The kit can include, for example, glucose-6-phosphate, nicotinamide adenine dinucleotide phosphate, optionally a buffer, optionally a surfactant, and instructions for preparing a reaction mixture that facilitates a reaction of nicotinamide adenine dinucleotide phosphate, glucose-6-phosphate and G6PDH. In some embodiments, the kit also includes an enzyme denaturant. In some embodiments, the kit further includes instructions for preparing a sample for analysis on a mass spectrometer. In further embodiments, the kit also includes a calibration curve which comprises a plot of [6-phosphogluconic acid/glucose-6-phosphate area] versus milliUnits of G6PDH.

These and other features of the applicant's teachings are set forth herein.

### DESCRIPTION OF THE DRAWINGS

FIG. 1 depicts a mass spectrometric reading in MRM (Multiple Reaction Monitoring) obtained from an exemplary method of the applicant's teachings.

FIG. 2 depicts comparative mass spectrometric readings in MRM obtained from exemplary methods of the applicant's teachings.

FIG. 3 depicts an exemplary standard curve obtained using various methods of the applicant's teachings.

FIGS. 4A and 4B depict mass spectrometric readings in MRM obtained from an exemplary method of the applicant's teachings.

FIG. 5 depicts an exemplary standard curve obtained using various methods of the applicant's teachings.

FIGS. 6A and 6B depict mass spectrometric readings in MRM obtained from an exemplary method of the applicant's teachings.

FIGS. 7A and 7B are a correlation plot and a Bland and Altman plot, respectively, comparing an exemplary method of the applicant's teachings with a commercially available assay.

### DESCRIPTION

The applicant's teachings are based, at least in part, on the finding that mass spectroscopy, e.g., MS/MS, LC/MS or LC/MS/MS, can be used to quickly and precisely determine an amount of G6PDH in a biological sample. Without wishing to be bound by any particular theory, it is believed that the ability to determine an amount of G6PDH (e.g., rather than merely its presence or absence) would be useful, for example in the detection of a partial G6PDH deficiency in a subject.

G6PDH converts glucose-6-phosphate to 6-phosphogluconolactone with the concurrent reduction of NADP to NADPH. 6-phosphogluconolactone is then converted to 6-phosphogluconic acid by gluconolactonase. Previous methods for determining G6PDH activity, including the Beutler method, exploit the formation of NADPH as the indicator of enzymatic reaction. However, amounts of NADPH may be difficult to determine in such an assay by mass spectroscopy because the C13-isotopomer of the NADP overlaps with the first C12 isotopomer of NADPH. Accordingly, amounts of 6-phosphogluconic acid are monitored in the methods provided herein.

Accordingly, in some embodiments, the applicant's teachings provide a high throughput method for detecting an amount of G6PDH in a biological sample. Such a method includes reacting a biological sample with G6P and NADP (e.g., in the presence of a surfactant and a buffer) under suitable conditions to form 6-phosphogluconic acid; quenching the biological sample to form a quenched sample; and detecting by mass spectrometry a presence or amount of 6-phosphogluconic acid in the quenched sample. In such a method, the amount of 6-phosphogluconic acid in the quenched sample is related to the amount of G6PDH in the biological sample.

As used herein, the term “high-throughput” refers to the process of assaying a large number of samples in a relatively short period of time. High-throughput assays may be accomplished using, for example, 96-, 384-, and 1536-well plates. In some embodiments, the aim of high-throughput methods is to screen samples at a rate that exceeds about 250 samples per week, about 300 samples per week, about 350 samples per week, about 400 samples per week, about 450 samples per week, about 500 samples per week, about 550 samples per week, about 600 samples per week, about 650 samples per week, about 700 samples per week, about 750 samples per week, about 800 samples per week, about 850 samples per week, about 900 samples per week, about 950 samples per week, about 1000 samples per week, about 1100 samples per week, about 1200 samples per week, about 1300 samples per week, about 1400 samples per week, about 1500 samples per week. In some embodiments, the aim of high-throughput methods is to screen samples at a rate that exceeds about 1000 samples per week.

In some embodiments, the methods described herein allow for detection of low levels of G6PDH. For example, the methods described herein allow for detection of about 1.00 milliUnits of G6PDH or less, about 0.90 milliUnits of G6PDH or less, about 0.80 milliUnits of G6PDH or less, about 0.70 milliUnits of G6PDH or less, about 0.60 milliUnits of G6PDH or less, about 0.50 milliUnits of G6PDH or less, about 0.45 milliUnits of G6PDH or less, about 0.40 milliUnits of G6PDH or less, about 0.35 milliUnits of G6PDH or less, about 0.30 milliUnits of G6PDH or less, about 0.25 milliUnits of G6PDH or less, about 0.20 milliUnits of G6PDH or less, about 0.15 milliUnits of G6PDH or less, about 0.10 milliUnits of G6PDH or less, about 0.05 milliUnits of G6PDH or less, about 0.04 milliUnits of G6PDH or less, about 0.03 milliUnits of G6PDH or less, about 0.02 milliUnits of G6PDH or less, or even about 0.01 milliUnits of G6PDH or less. In some embodiments, the methods described herein allow for detection of about 0.10 milliUnits of G6PDH or less. In some embodiments, the methods described herein allow for detection of about 0.05 milliUnits of G6PDH or less. In some embodiments, the methods described herein allow for detection of about 0.01 milliUnits of G6PDH or less. In some embodiments, the methods described herein allow for detection of levels of G6PDH of at least about 0.01 milliUnits of G6PDH. In some embodiments, the methods described herein allow for detection of levels of G6PDH of at least 0.01 milliUnits of G6PDH.

In some embodiments, methods are provided herein where the detection of an amount of G6PDH is not substantially affected by fluctuations in temperature, pH and/or salt concentration. In some embodiments, methods are provided herein where the detection of an amount of G6PDH is not substantially affected by fluctuations in temperature. In some embodiments, methods are provided herein where the detection of an amount of G6PDH is not substantially affected by fluctuations in temperature during detection. In some embodiments, methods are provided herein where the detection of an amount of G6PDH is not substantially affected by fluctuations in temperature during storage of the sample.

In some embodiments, an internal standard is used to produce a more accurate measurement of the amount of 6-phosphogluconic acid detected in the sample. In some embodiments, the internal standard is a compound/substance added to the sample which does not react with other substances in the sample, and thus provides a constant value. In some embodiments, the internal standard is a level of one of the other compounds/substances naturally occurring in the sample. In some embodiments, glucose-6-phosphate can be used as an internal standard.

As used herein, the term “biological sample” refers, without limitation, to a sample of a host body. Biological samples useful in the applicant's teachings may be fresh (e.g., drawn within a few hours prior to analysis), refrigerated or frozen (e.g., drawn and placed into a refrigerator or freezer until analysis), or dried (e.g., drawn and placed on Whatman paper or spotted directly on Whatman paper). Exemplary biological samples include, for example, blood, interstitial fluid, spinal fluid, saliva, urine, tears, sweat, or the like. Additional biological samples include, but are not limited to, cells or tissues, or cultures (or subcultures) thereof, crude or processed cell lysates (including whole cell lysates), body fluids, tissue extracts or cell extracts, feces, cerebral fluid, amniotic fluid, lymph fluid or a fluid from a glandular secretion. The sample may be processed prior to contact with a substrate described herein by any method known in the art. For example, the sample may be subjected to a precipitation step, column chromatography step, heat step, etc. If the sample contains other enzymes that may interfere with the activity of the G6PDH, an inactivating agent (e.g., an active site directed irreversible inhibitor) can be added to the sample to inactivate the activity that is not desired. In some embodiments, the biological sample is a blood sample. In some embodiments, the biological sample is a dried blood sample.

“Detect” and “detection” are intended to encompass detection, measurement and/or characterization of G6PDH enzyme or its enzyme activity. For example, enzyme activity may be detected in the course of screening for, detecting or characterizing modulators of the enzyme activity.

In some embodiments, detecting the amount of G6PDH occurs in less than about 10 minutes. In some embodiments, detecting the amount of G6PDH occurs in less than about 7.5 minutes. In some embodiments, detecting the amount of G6PDH occurs in less than about 5 minutes. In some embodiments, detecting the amount of G6PDH occurs in less than about 4 minutes. In some embodiments, detecting the amount of G6PDH occurs in less than about 3 minutes. In some embodiments, detecting the amount of G6PDH occurs in less than about 2.5 minutes. In some embodiments, detecting the amount of G6PDH occurs in less than about 2 minutes.

As used herein, the term “quench” refers to inactivating a reagent or halting a chemical reaction. It is to be understood that quenching refers to the inactivation or halting regardless of the mechanism by which the inactivation or halting is achieved. As specific non-limiting examples, the quenching may be due to changes in pH or to the addition of a quenching agent, which may react with the G6PDH or G6P, such that it is no longer available for reaction. Thus, a quenched sample refers to a sample in which G6PDH or G6P are no longer undergoing a reaction. The term “quenching reagent” as used herein is any reagent which is able to interact with a G6PDH or G6P such that the G6PDH or G6P are quenched and therefore unable to further react. In some embodiments, the quenching agent is an enzyme denaturant. For example, in some embodiments, the quenching agent denatures the G6PDH. Enzyme denaturants typically trigger a non-covalent change in the structure (e.g., secondary, tertiary or quaternary structure) of the enzyme. Examples of quenching reagents include, but are not limited to, acetonitrile, methanol, and mixtures thereof.

The term “buffer” refers to a solution, typically consisting of a weak acid and its conjugate base or a weak base and its conjugate acid, wherein the pH of the solution changes very little upon additions of small amounts of acid or base. Suitable buffers include those described in the “Biological Buffers” section of the Sigma Catalog, as well as online at sigmaaldrich.com. Exemplary buffers include, but are not limited to, potassium phosphate buffers, MES, MOPS, HEPES, Tris (Trizma), bicine, TAPS, CAPS, and the like. The buffer is present in an amount sufficient to generate and maintain a desired pH. For example, the pH can be from 2 to 12, from 4 to 11, or from 6 to 10.

The term “surfactant” refers to a molecule having a polar head group, which typically energetically prefers solvation by water, and a hydrophobic tail which is typically not well solvated by water. The surfactant may be ionic (i.e., anionic, cationic) or nonionic. Exemplary surfactants include, but are not limited to, polysorbates/Tweens (e.g., Tween 20 and Tween 80), pluronics (e.g., F68 and F88), Tritons (e.g., TRITON® X-114, X-102, X-45, X-15), poloxamers (e.g., poloxamer 188), sorbitan esters or Spans (e.g., Span 20 and Span 80), lipids (e.g., phospholipids, lecithin, phosphatidylcholines, phosphatidylethanolamines), alcohol ethoxylates (e.g., BRIJ® 56, C₁₆H₃₃(OCH₂CH₂)₁₀OH, BRIJ® 58, C₁₆H₃₃(OCH₂CH₂)₂₀OH), fatty acids and fatty esters, steroids (e.g., cholesterol), chelating agents (e.g., EDTA), amine ethoxylates, glucosides, glucamides, polyalkyleneoxides, sodium dodecyl sulfate (SDS), sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, lauroamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, MONAQUAT™ surfactants, polyethylene glycol, polypropylene glycol, and copolymers of ethylene and propylene glycol. Other surfactants may be found, for example, in McCutcheon's Emulsifiers and Detergents, Manuf. Confectioners Pub. Co., Glen Rock, N.J., 2000 and in Kirk-Othmer, Encyclopedia of Chemical Technologies, 2nd Edition, Vol. 19, pages 512-564.

In some embodiments, the G6PDH is contacted with the G6P for a reaction time of at least about 15 seconds, about 30 seconds, about 45 seconds, 1 minute, 2 minutes, 3 minutes, 4 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some embodiments, the G6PDH is contacted with the G6P for a reaction time at least about 15 minutes. In some embodiments, the G6PDH is contacted with the G6P for a reaction time at least about 30 minutes. In some embodiments, the G6PDH is contacted with the G6P for a reaction time at least 15 minutes. In some embodiments, the G6PDH is contacted with the G6P for a reaction time at least 30 minutes.

The methods of the applicant's teachings can be practiced using tandem mass spectrometers and other mass spectrometers that have the ability to select and fragment molecular ions. Tandem mass spectrometers (and, to some degree, single-stage mass spectrometers) have the ability to select and fragment molecular ions according to their mass-to-charge (m/z) ratio, and then record the resulting fragment (daughter) ion spectra. More specifically, daughter fragment ion spectra can be generated by subjecting selected ions to dissociative energy levels (e.g. collision-induced dissociation (CID)). For example, ions corresponding to compounds of a particular m/z ratio can be selected from a first mass analysis, fragmented and reanalyzed in a second mass analysis. Representative instruments that can perform such tandem mass analysis include, but are not limited to, magnetic four-sector, tandem time-of-flight, triple quadrupole, ion-trap, and hybrid quadrupole time-of-flight (Q-TOF) mass spectrometers.

These types of mass spectrometers may be used in conjunction with a variety of ionization sources, including, but not limited to, electrospray ionization (ESI) and matrix-assisted laser desorption ionization (MALDI). Ionization sources can be used to generate charged species for the first mass analysis where the analytes do not already possess a fixed charge. Additional mass spectrometry instruments and fragmentation methods include post-source decay in MALDI-MS instruments and high-energy CID using MALDI-TOF-TOF MS. A review of tandem mass spectrometers can be found, for example, in R. Aebersold and D. Goodlett, Mass Spectrometry in Proteomics. Chem. Rev. 101: 269 295 (2001).

In some embodiments, the mass spectrometry is tandem mass spectrometry. In some embodiments, the mass spectrometry is accomplished using a mass spectrometer equipped with a thermally assisted electrospray ionization probe (e.g., TurboIonSpray® (TIS), Turbo-V, or similar). In some embodiments, the mass spectrometry utilizes multiple reaction monitoring (MRM). Exemplary methods for generating mass spectra may be found, for example, in U.S. Pat. No. 6,930,305 and U.S. Pat. No. 7,145,133, both of which are incorporated herein by this reference. In some embodiments, the mass spectrometry utilizes an AB Sciex API 4000™ tandem mass spectrometer.

In some embodiments, the processing of a sample can involve separation, e.g., prior to mass analysis. For example, components of the sample can be separated and mass analysis performed on only a fraction of the sample mixture. In this way, the complexity of the analysis can be reduced because separated analytes can be individually analyzed. Separation can be performed by chromatography. For example, liquid chromatography/mass spectrometry (LC/MS) or liquid chromatography/tandem mass spectrometry (LC/MS/MS) can be used to effect such a sample separation and mass analysis. Moreover, any chromatographic separation process suitable to separate the analytes of interest can be used. For example, the chromatographic separation can be normal phase chromatography, reversed-phase chromatography, ion-exchange chromatography, size exclusion chromatography or affinity chromatography. Separation can also be performed electrophoretically. Non-limiting examples of electrophoretic separation techniques include, but are not limited to, 1D electrophoresis, 2D electrophoresis and/or capillary electrophoresis. In some embodiments, the mass spectrometry provided herein is accomplished using a mass spectrometer equipped with a reversed phase liquid chromatography column.

Also provided herein are methods for detecting G6PDH deficiency in a subject. Such a method includes determining by mass spectrometry an amount of G6PDH in a biological sample from a subject; and comparing the amount of G6PDH in the biological sample to an appropriate control, wherein G6PDH deficiency is detected when the amount of G6PDH in the sample is less than the appropriate control. G6PDH deficiency is generally an X-linked, hereditary genetic defect, typically due to a mutation in the G6PDH gene. This deficiency is common, being present in more than 400 million people (typically of African, Middle Eastern and South Asian ancestry) worldwide.

The G6PDH gene is found on the long arm of the X chromosome (band Xq28) and spans approximately 18.5 kilobases. In some embodiments, the G6PDH deficiency is due to a G6PDH gene mutation. G6PDH gene mutations can be found, for example, in Beutler E., “G6PD Deficiency” Blood, 84(11): 3613-3636, (1994). In some embodiments, the G6PDH deficiency is due to one of the gene mutations listed in Table 1.

TABLE 1
Exemplary G6PDH mutations/variants

| Variants/mutations | | | | | |
|---|---|---|---|---|---|
| | Isoform | | | | |
| Desig- | G6PD- | Gene | | | Protein |
| nation | Protein | Type | Subtype | Position | Structure change |
| G6PD-A(+) | G6PD A | Polymorphism | A→G | 376 | Asparagine→Aspartic |
| | | nucleotide | | (Exon 5) | acid (ASN126ASP) |
| G6PD-A(−) | G6PD A | Substitution | G→A | 376 | Valine→Methionine |
| | | nucleotide | | (Exon 5) | (VAL68MET) |
| | | | | and 202 | Asparagine→Aspartic |
| | | | | | acid (ASN126ASP) |
| G6PD- | G6PD B | Substitution | C→T | 563 | Serine→Phenylalanine |
| Mediterran | | nucleotide | | (Exon 6) | (SER188PHE) |
| G6PD- | G6PD B | Substitution | G→T | 1376 | Arginine→Leucine |
| Canton | | nucleotide | | | (ARG459LEU) |
| G6PD- | G6PD | Substitution | G→A | 1003 | Alanine→Threonine |
| Chatham | | nucleotide | | | (ALA335THR) |
| G6PD- | G6PD B | Substitution | G→A | 1376 | Arginine→Proline |
| Cosenza | | nucleotide | | | (ARG459PRO) |
| G6PD- | G6PD | Substitution | G→A | 487 | Glycine→Serine |
| Mahidol | | nucleotide | | (Exon 6) | (GLY163SER) |
| G6PD- | G6PD | Substitution | | | Alanine→Glycine |
| Orissa | | nucleotide | | | (ALA44GLY) |
| G6PD- | G6PD A− | Substitution | A→G ± | 376 | Asparagine→Aspartic |
| Asahi | | nucleotide | G→A | (Exon 5) | acid (ASN126ASP) |
| | | (several) | | 202 | Valine→Methionine |
| | | | | | (VAL68MET) |

In some embodiments, G6PDH deficiency in a subject is indicated by an amount of G6PDH in a biological sample from the subject which is about 5% less than the control, about 10% less than the control, about 15% less than the control, about 20% less than the control, about 25% less than the control, about 30% less than the control, about 35% less than the control, about 40% less than the control, about 45% less than the control, about 50% less than the control, about 55% less than the control, about 60% less than the control, about 65% less than the control, about 70% less than the control, about 75% less than the control, about 80% less than the control, about 85% less than the control, about 90% less than the control, about 95% less than the control, or about 100% less than the control. For example, in some embodiments, G6PDH deficiency in a subject is indicated by an amount of G6PDH in a biological sample from the subject which is at least about 10% less than the control. In some embodiments, G6PDH deficiency in a subject is indicated by an amount of G6PDH in a biological sample from the subject which is at least about 25% less than the control. In some embodiments, G6PDH deficiency in a subject is indicated by an amount of G6PDH in a biological sample from the subject which is at least about 50% less than the control. For example, in some embodiments, G6PDH deficiency in a subject is indicated by an amount of G6PDH in a biological sample from the subject which is at least 10%, at least 25%, or at least 50% less than the control.

As used herein, the term “subject” refers to animals such as mammals, including, but not limited to, humans, primates, cows, sheep, goats, horses, pigs, dogs, cats, rabbits, guinea pigs, rats, mice or other bovine, ovine, equine, canine, feline, rodent or murine species. In some embodiments, the subject is a human. In some embodiments, the subject is a neonate or an infant. In some embodiments, the subject is a pregnant woman.

In some embodiments, the applicant's teachings provide methods for screening compounds for the ability to modulate G6PDH. In some embodiments, the effects of modulators of G6PDH activity (e.g., enhancers or inhibitors) can be studied using the methods described herein. Furthermore, substrate specificities of G6PDH can also be studied using the methods described herein.

In some embodiments, methods for diagnosing G6PDH-associated disorders are described herein. As used herein, the term “G6PDH-associated disorders” refers to diseases and/or disorders which are at least partially attributable to a deficiency of G6PDH. In some embodiments, G6PDH-associated disorders include diseases and/or disorders which are attributable to a deficiency of G6PDH of at least about 10%, at least about 20%, at least about 30%, at least about 40%, at least about 50%, at least about 60%, at least about 70%, at least about 80%, at least about 90%, about 100%. In some embodiments, G6PDH-associated disorders include diseases and/or disorders which are attributable to a deficiency of G6PDH of at least 25%. In some embodiments, G6PDH-associated disorders include diseases and/or disorders which are attributable to a deficiency of G6PDH of at least 50%. In some embodiments, G6PDH-associated disorders include diseases and/or disorders which are attributable to a deficiency of G6PDH of at least 75%.

In some embodiments, the G6PDH-associated disorder is selected from chronic hemolysis, acute hemolytic anemia, favism, neonatal hyperbilirubinemia and pre-eclampsia. Other G6PDH-associated disorders may include, for example, platelet abnormalities, skin pedicle flap loss, impairment of athletic performance, seizure disorders, increased incidence of cataracts, schizophrenia or depression, impaired renin release, diabetes, abnormal insulin release, cardiovascular disease, cholelithiasis, myoglobuinuria, increased susceptibility to infection, abnormal leukocyte function, increased jaundice in hepatitis, mental retardation or increased serum dehydroepiandrostrone sulfate. See, e.g., Beutler, E., “G6PD Deficiency” Blood, 84(11): 3613-3636, (1994).

In some embodiments, the G6PDH-associated disorder is a disorder caused or induced by a substance capable of inducing G6PDH deficiency. Such substances include, but are not limited to, fava beans, antimalarial drugs such as primaquine, pamaquine and chloroquine, sulfonamides such as sulfanilamide, sulfamethoxazole and mafenide, nitrofurantoin, acetylsalicylic acid, acetophenetidine, thiazolesulfone, methylene blue, naphthalene, analgesics such as aspirin, phenazopyridine and acetanilide, and a few non-sulfa antibiotics such as nalidixic acid, nitrofurantoin, isoniazid and furazolidone.

In some embodiments, methods for diagnosing acute hemolytic anemia in a subject are provided herein. Such methods include detecting G6PDH deficiency in a subject in accordance with the methods described above, wherein a G6PDH deficiency in the subject indicates acute hemolytic anemia in the subject.

In some embodiments, an amount of G6PDH in the sample which is at least about 90% less than the control indicates acute hemolytic anemia in the subject. In some embodiments, an amount of G6PDH in the sample which is at least about 80% less than the control indicates acute hemolytic anemia in the subject. In some embodiments, an amount of G6PDH in the sample which is at least about 70% less than the control indicates acute hemolytic anemia in the subject. In some embodiments, an amount of G6PDH in the sample which is at least about 60% less than the control indicates acute hemolytic anemia in the subject. In some embodiments, an amount of G6PDH in the sample which is at least about 50% less than the control indicates acute hemolytic anemia in the subject. In some embodiments, an amount of G6PDH in the sample which is at least about 40% less than the control indicates acute hemolytic anemia in the subject. In some embodiments, an amount of G6PDH in the sample which is at least about 30% less than the control indicates acute hemolytic anemia in the subject. In some embodiments, an amount of G6PDH in the sample which is at least about 20% less than the control indicates acute hemolytic anemia in the subject. In some embodiments, an amount of G6PDH in the sample which is at least about 10% less than the control indicates acute hemolytic anemia in the subject.

In some embodiments, an amount of G6PDH in the sample which is 75% less than the control indicates acute hemolytic anemia in the subject. In some embodiments, an amount of G6PDH in the sample which is 50% less than the control indicates acute hemolytic anemia in the subject. In some embodiments, an amount of G6PDH in the sample which is 25% less than the control indicates acute hemolytic anemia in the subject.

In some embodiments, methods for diagnosing pre-eclampsia in a subject are provided herein. Such methods include detecting G6PDH deficiency in a subject in accordance with the methods described above, wherein a G6PDH deficiency in the subject indicates pre-eclampsia in the subject.

In some embodiments, an amount of G6PDH in the sample which is at least about 90% less than the control indicates pre-eclampsia in the subject. In some embodiments, an amount of G6PDH in the sample which is at least about 80% less than the control indicates pre-eclampsia in the subject. In some embodiments, an amount of G6PDH in the sample which is at least about 70% less than the control indicates pre-eclampsia in the subject. In some embodiments, an amount of G6PDH in the sample which is at least about 60% less than the control indicates pre-eclampsia in the subject. In some embodiments, an amount of G6PDH in the sample which is at least about 50% less than the control indicates pre-eclampsia in the subject. In some embodiments, an amount of G6PDH in the sample which is at least about 40% less than the control indicates pre-eclampsia in the subject. In some embodiments, an amount of G6PDH in the sample which is at least about 30% less than the control indicates pre-eclampsia in the subject. In some embodiments, an amount of G6PDH in the sample which is at least about 20% less than the control indicates pre-eclampsia in the subject. In some embodiments, an amount of G6PDH in the sample which is at least about 10% less than the control indicates pre-eclampsia in the subject.

In some embodiments, an amount of G6PDH in the sample which is 75% less than the control indicates pre-eclampsia in the subject. In some embodiments, an amount of G6PDH in the sample which is 50% less than the control indicates pre-eclampsia in the subject. In some embodiments, an amount of G6PDH in the sample which is 25% less than the control indicates pre-eclampsia in the subject.

In some embodiments, prognostic methods for predicting increased mortality and/or morbidity resulting from pre-eclampsia or acute hemolytic anemia in a subject are provided herein. Such methods include detecting G6PDH deficiency in a subject in accordance with the methods described above, wherein an amount of the G6PDH in the biological sample which is 50% or less than the control indicates a prognosis of increased mortality and/or morbidity in the subject. In some embodiments, an amount of the G6PDH in the biological sample which is 75% or less than the control indicates a prognosis of increased mortality and/or morbidity in the subject.

As used herein, the term “appropriate control” is a control based upon G6PDH levels in a normal population. That is, in some embodiments, the appropriate control is a predetermined value. In some embodiments, the appropriate control is a level detected in a single individual known to have normal G6PDH activity. In some embodiments, the appropriate control is an average level from a population of individuals having normal G6PDH activity. In some embodiments, the appropriate control is a level detected in a patient prior to administration of a substance capable of inducing a G6PDH deficiency.

In some embodiments, the diagnostic or prognostic methods provided herein include any one of the following steps: reacting the biological sample with G6P and NADP in the presence of a surfactant and a buffer under suitable conditions to form 6-phosphogluconic acid; quenching the biological sample to form a quenched sample; and detecting by mass spectrometry a presence or amount of 6-phosphogluconic acid in the quenched sample. In such methods, the amount of 6-phosphogluconic acid in the quenched sample is related to the amount of G6PDH in the biological sample.

Also provided are kits for performing the methods described herein. In some embodiments, the kit comprises glucose-6-phosphate, nicotinamide adenine dinucleotide phosphate, a surfactant and a buffer for preparing a reaction mixture that facilitates the reaction of nicotinamide adenine dinucleotide phosphate, glucose-6-phosphate and G6PDH. The buffer and/or the surfactant may be provided in a container in dry form or liquid form. Exemplary suitable buffers and surfactants are provided hereinabove. The buffer is typically present in the kit at least in an amount sufficient to produce a particular pH in the mixture. In some embodiments, the buffer is provided as a stock solution having a pre-selected pH and buffer concentration. In some embodiments, acids and/or bases are also provided in the kit in order to adjust the reaction mixture to a desired pH. The kit may additionally include a quenching solution, e.g., an enzyme denaturant, such as an acetonitrile:methanol mixture. The kit may additionally include one or more one or more diluents, e.g., solvents suitable for use in a mass spectroscopy system. The kit may additionally include other components that are beneficial to enzyme activity, such as salts (e.g., KCl, NaCl, or NaOAc), metal salts (e.g., Ca²⁺ salts such as CaCl₂, MgCl₂, MnCl₂, ZnCl₂, or Zn(OAc), and/or other components that may be useful for the G6PDH enzyme. These other components can be provided separately from each other or mixed together in dry or liquid form.

The glucose-6-phosphate and/or nicotinamide adenine dinucleotide phosphate can be provided in dry or liquid form, together with or separate from the buffer. To facilitate dissolution in the reaction mixture, the glucose-6-phosphate and/or nicotinamide adenine dinucleotide phosphate can be provided in an aqueous solution, partially aqueous solution, or non-aqueous stock solution that is miscible with the other components of the reaction mixture.

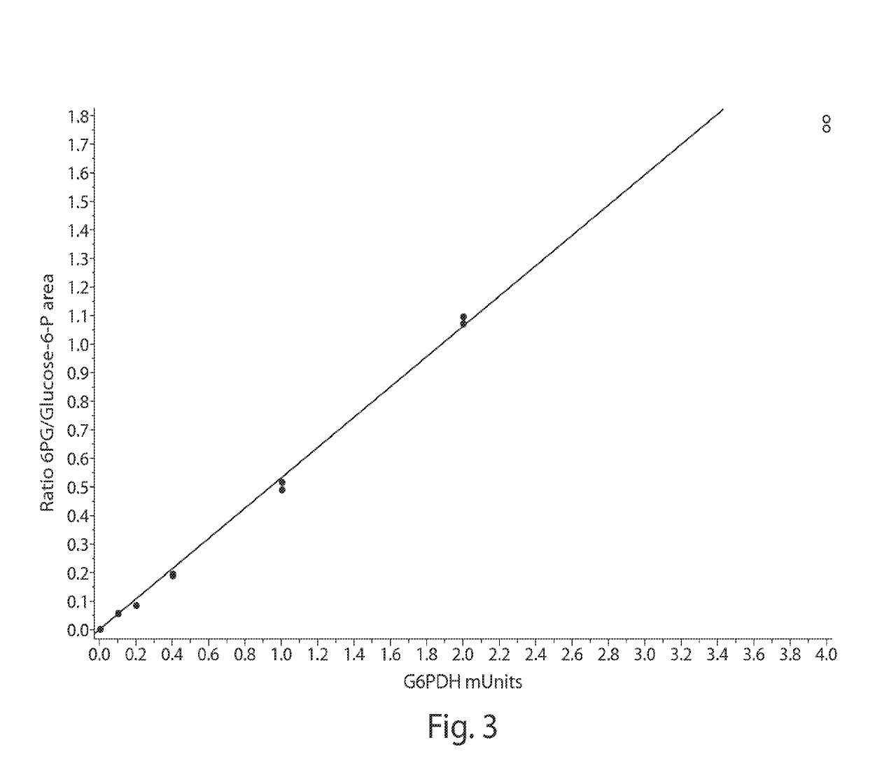

In some embodiments, the kit further includes instructions for use. For example, the kit can include instructions for preparing a reaction mixture that facilitates a reaction of nicotinamide adenine dinucleotide phosphate, glucose-6-phosphate and G6PDH. In some embodiments, the kit includes instructions for preparing a sample for analysis on a mass spectrometer. In some embodiments, the kit further comprises a pre-prepared calibration curve, such as the curve shown in FIG. 3, which would allow a user to determine an amount of G6PDH in the sample based upon an amount of 6-phosphogluconic acid determined using a mass spectrometer. For example, the pre-prepared calibration curve may be a plot of [6-phosphogluconic acid/glucose-6-phosphate area] versus milliUnits of G6PDH. In some embodiments, the kit includes standard samples, with pre-determined amounts of G6PDH, such that a user may produce their own calibration curve. In some embodiments, the kit further includes instructions on how to prepare a calibration curve. The instructions may include any combination of:
a. Mixing the glucose-6-phosphate and nicotinamide adenine dinucleotide phosphate in the presence of a buffer and/or a surfactant for a set time period (e.g., 15 minutes, 20 minutes, 25 minutes, 30 minutes, etc.);
b. Quenching the sample using a quenching solution;
c. Analyzing the quenched sample using a mass spectrometer;
d. Preparing a calibration curve;
e. Comparing the analysis (e.g, the mass spectrum) of the quenched sample with a calibration curve.

The operation of the various compositions and methods can be further understood in light of the following non-limiting examples, which should not be construed as limiting the scope of the applicant's teachings in any way.

### EXEMPLIFICATION

### Example 1

### DBS Sample

Paper filters with Dried Blood Spots (DBS) from a Neonatal screening program were utilized in the present example. A 3-mm diameter disc was removed from the filter paper and the blood sample on this disc was added using 50 μL of “Reagent 1” to a 1.5-mL Eppendorf tube. “Reagent 1” was formulated by mixing 3 volumes of bi-distilled water, 3 volumes of 250 mM K₂HPO₄, 2 volumes of 1% Saponine for Molecular Biology, 1 volume of 7.5 mM Nicotinamide Adenine Dinucleotide Phosphate and 1 volume 10 mM Glucose-6-Phosphate. The Eppendorf tube was closed, and after gentle mixing, the tube was placed in an incubation oven at 37° C. for 30 minutes. The reaction was quenched by adding 100 μL of a 1:1 Acetonitrile:Methanol (LC-MS grade) mixture. After centrifugation, 10 μL clear supernatant was diluted with 240 μL of 20 mM Butyl-Dimethyl-Ammonium Bicarbonate (BDMAB) in water.

2 μL of this solution were injected into the LC/MS/MS system, which included an Agilent 1200 LC system equipped with a Phenomenex C18 column and an AB Sciex API 4000™ Tandem mass spectrometer equipped with the TIS probe. The liquid chromatography was performed by injecting the sample on the column kept at 50° C. and flowed by 300 μL/min of the LC-eluent (20 mM BDMAB+3% MetOH) in isocratic mode and for 2.5 minutes. The mass spectrometer was operated in MRM, Negative Ion Mode with the TIS at −4500 Volts and with the Nominal Gun temperature at 350° C. Exploited transitions are as follows:

259.2>79.0 for Glucose-6-Phosphate with a DP: −60 V and a CE: −70 eV 275.2>79.0 for 6-Phospho-Gluconic acid with a DP: −60V and a CE: −70 eV 742.1>79.0 for the NADP with a DP: −80 V and a CE: −130 eV.

In “Quant” or “MultiQuant” modes, the traces corresponding to the transitions 275.2>79.0 and 259.2>79.0 were processed as the analyte (6-Phosphogluconic acid) and as the internal standard (G6P), respectively. The obtained ratio was compared with a calibration curve, obtained using commercially available G6PDH, as described in Example 2.

The transition 742.1>79.0, which monitored NADP, was used as an internal check for false positive results, because enzyme deficiency would be accompanied by a high signal for NADP. Thus, where both the traces for 6-Phosphogluconic acid and for NADP were low or absent, the results are considered false.

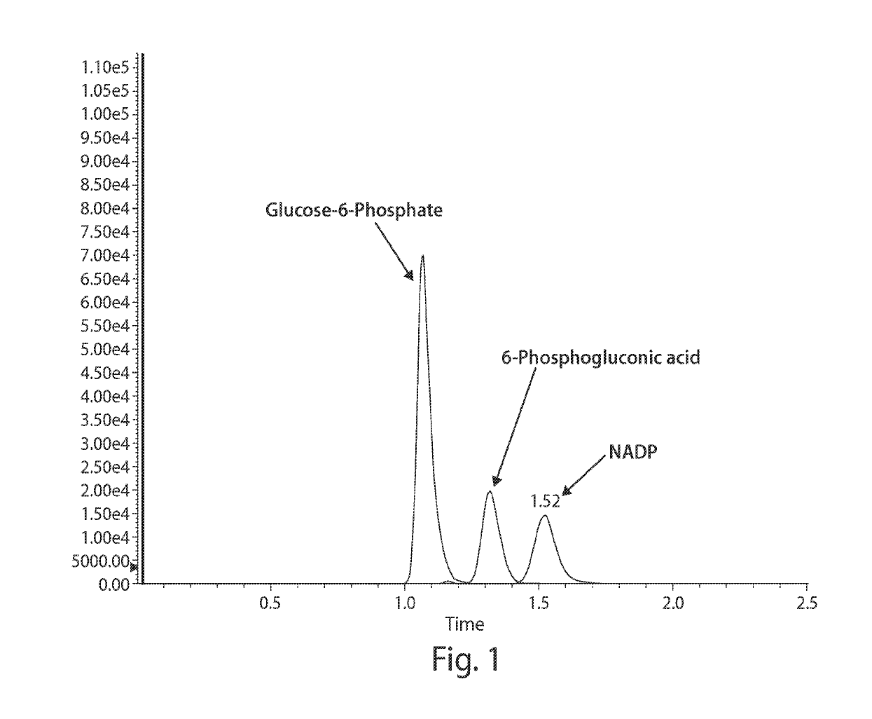

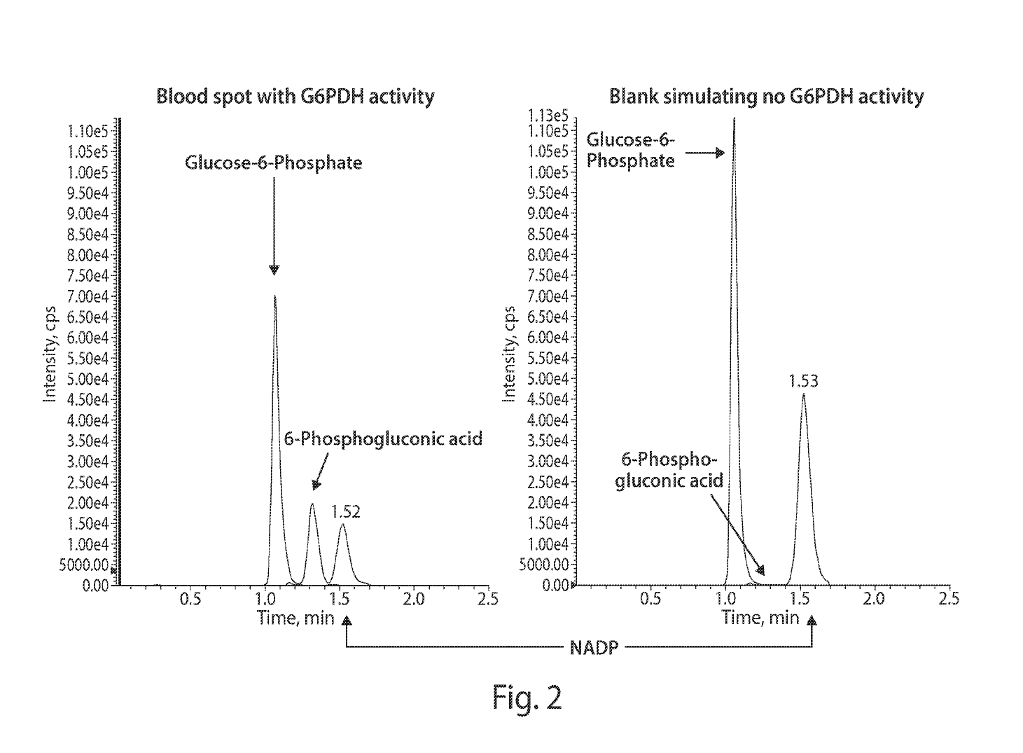

FIG. 1 shows an exemplary mass spectrometric reading in MRM from a dried blood sample, showing levels of G6P, 6-phosphogluconic acid and NADP. FIG. 1 is representative of a normal level of G6PDH. FIG. 2 compares a normal sample with a sample having no G6PDH activity.

### Example 2

### Enzyme Calibration

A commercially-available enzyme standard (e.g., Sigma #G5885) was dissolved to obtain 100 milliUnits/mL with a solution of 50% bi-distilled water, 30% 250 mM K₂HPO₄ and 20% of 1% Saponine for Molecular Biology. 0, 1, 2, 4, 10, 20 and 40 μL of this solution (corresponding to 0, 0.1, 0.2, 0.4, 1.0, 2.0 and 4.0 milliUnits of enzyme) were added to 50 μL of “Reagent 1” (described in Example 1) in a 1.5-mL Eppendorf tube. The Eppendorf tube was closed, and after gentle mixing, the tube was placed in an incubation oven at 37° C. for 30 minutes. The reaction was quenched by adding 100 μL of a 1:1 Acetonitrile:Methanol (LC-MS grade) mixture. After centrifugation, 10 μL clear supernatant was diluted with 240 μL of 20 mM Butyl-Dimethyl-Ammonium Bicarbonate (BDMAB) in water. 2 μL of this solution were injected into the LC/MS/MS system, as in Example 1. In “Quant” or “MultiQuant” modes, the traces corresponding to the transitions 275.2>79.0 and 259.2>79.0 were processed as for “analyte” and for “internal standard”, respectively. Quantitative assessment of specimens are made through the absolute reading in milliUnits and referenced to the amount of blood on the punched disk (3.1 μL). As a reference, an enzymatic activity around 3 milliUnits/DBS was retained as normal. Results are shown below in Table 2.

TABLE 2

| | 0.0 mU | 0.1 mU | 0.2 mU | 0.4 mU | 1.0 mU | 2.0 mU | 4.0 mU |
|---|---|---|---|---|---|---|---|
| Expected | 0.000000 | 0.100000 | 0.200000 | 0.400000 | 1.000000 | 2.000000 | 4.000000 |
| conc. | | | | | | | |
| # tested | 3 | 3 | 3 | 3 | 3 | 3 | 0 |
| Mean | 0.000 | 0.102329 | 0.162332 | 0.359533 | 0.936305 | 2.043591 | N/A |
| Std. Dev. | 0.000 | 0.004045 | 0.011148 | 0.006903 | 0.033458 | 0.024109 | N/A |
| % CV | N/A | 3.952955 | 6.867237 | 1.920072 | 3.573362 | 1.179734 | N/A |
| Accuracy | N/A | 102.329354 | 81.165960 | 89.883345 | 93.630452 | 102.179570 | N/A |

Based upon the actual blood volume sampled, the detectability of these methods appears to be about 1/300th of the average enzyme activity of normal samples. A linearity up to about 3-4 milliUnits/spot has been observed (see FIG. 3), but can be extended by decreasing the incubation time, if necessary.

### Example 3

### Analysis of Dried Blood Samples

A correlation study was organized utilizing anonymous excess samples from routine analysis in a hospital, previously collected and used in accordance with local ethical committee guidelines. Blood samples were spotted on a Guthrie card, dried and sent for measurement by LC/MS/MS. Analysis via LC/MS/MS was carried out as described above for Example 1. As a reference, samples were previously measured with the BioVision Kit (Catalog #K757-100 from BioVision Research Products, 980 Linda Vista Avenue, Mountain View, Calif. 94043 USA), as implemented in a routine hospital laboratory.

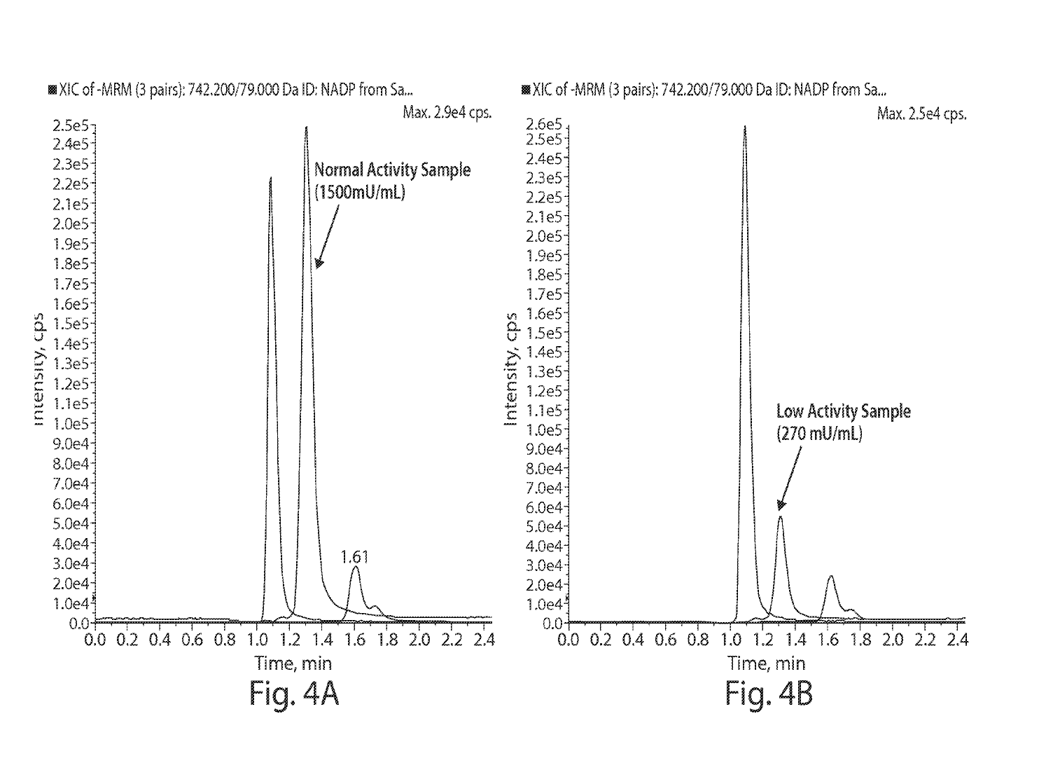

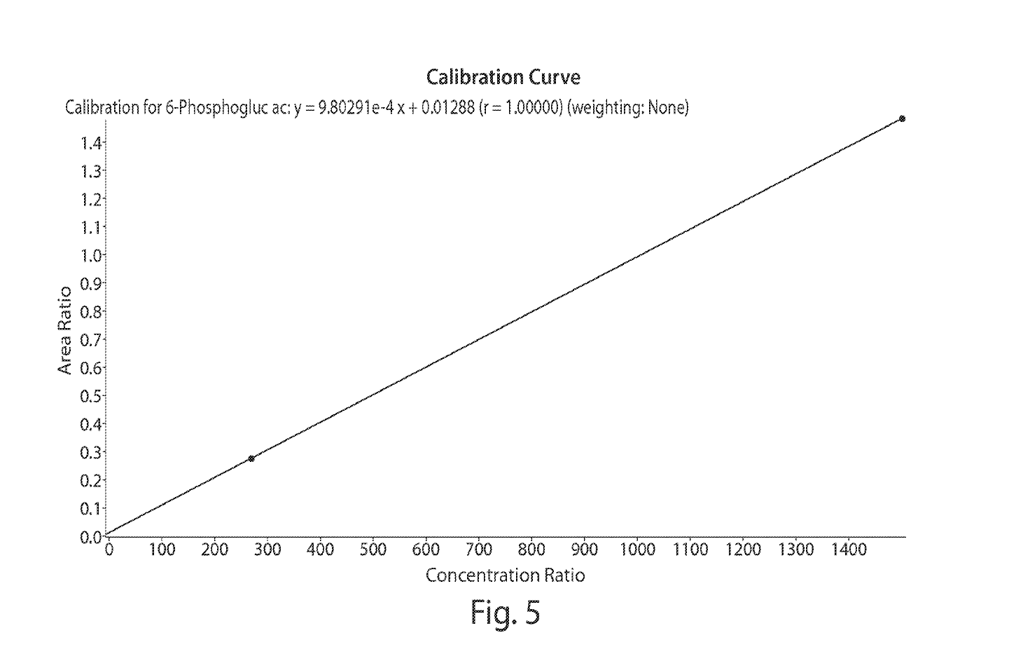

FIGS. 4A and 4B show two exemplary mass spectrometric readings in MRM from two dried blood samples, representing high and low G6PDH activity as measured by the BioVision Kit (BV). As can be seen by these Figures, LC-MS/MS is capable of measuring both high and low G6PDH activity. Because there is no homogeneous calibration available for both the assays, the calibration curve for the LC-MS/MS assay was generated using the above samples, which represented extreme activity values. The calibration used as standard for the LC-MS/MS is shown in FIG. 5.

The results from analysis of the samples are provided in Table 3, which also provides a listing of the values obtained using the BV Kit. G6PDH activity is expressed in milliUnits/mL (mU/mL) for both LC/MS/MS and the BV kit, with no normalization on hemoglobin for LC/MS/MS.

TABLE 3

| | BioVision | LC-MS/MS |
|---|---|---|
| Sample | (mU/mL) | (mU/mL) |
| n1 | 1100 | 1778 |
| n2 | 1230 | 1532 |
| n3 | 1230 | 1202 |
| n4 | 964 | 1848 |
| n5 | 1200 | 1796 |
| n6 | 1200 | 1865 |
| n7 | 1150 | 1757 |
| n8 | 20 | 40 * |
| n9 | 1065 | 1389 |
| n10 | 18 | 31 * |
| n11 | 1040 | 1429 |
| n12 | 1200 | 1992 |
| n13 | 1320 | 2032 |
| n14 | 987 | 1901 |
| n15 | 1110 | 1909 |
| n16 | 1190 | 1669 |
| n17 | 270 | 229 * |
| n18 | 1500 | 1496 |

* Samples n8, n10 and n17 are considered low activity samples.

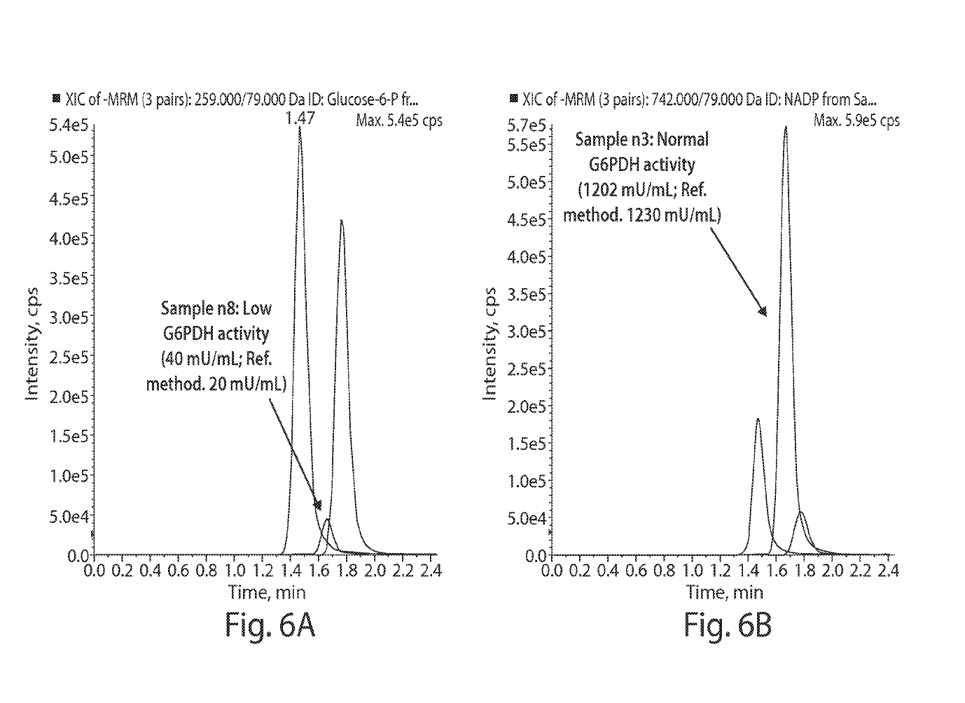

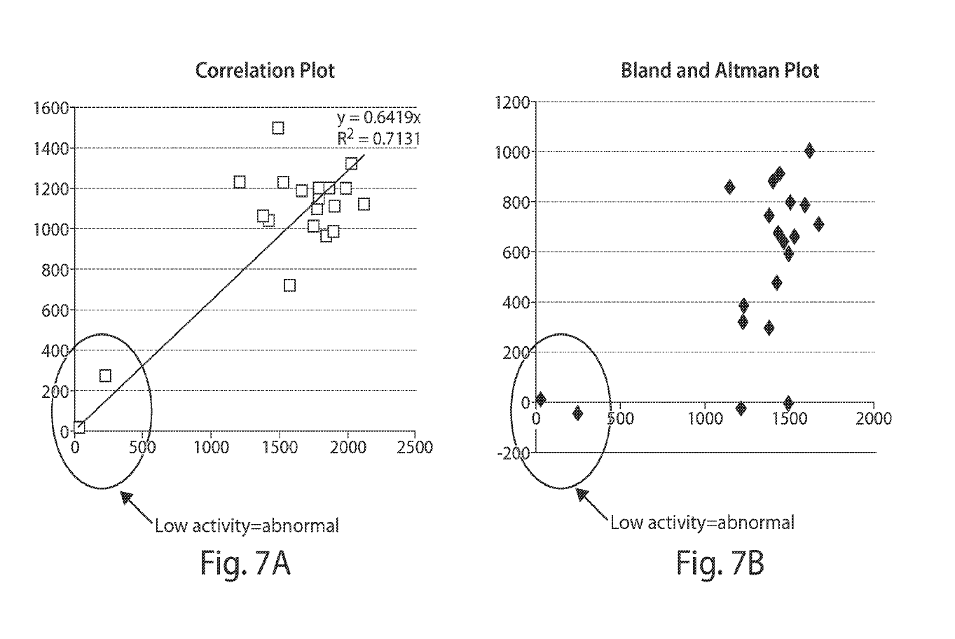

FIGS. 6A and 6B depict two exemplary mass spectrometric readings in MRM from two dried blood samples read as unknowns. These readings represent low and high G6PDH activity, with the LC/MS/MS assay giving measurements of 40 and 1202 milliUnits/mL, respectively, and the BV assay giving measurements of 20 and 1230 mU/mL, respectively. Additionally, FIGS. 7A and 7B are the correlation plot and the Bland and Altman plot (measures the agreement between two methods of clinical measurement) generated using the values measured in the present study. As can be seen by the above data and plots, there is good agreement between the values obtained using LC/MS/MS and those obtained using the BV kit, particularly with regard to the low activity samples.

### EQUIVALENTS

Those skilled in the art will recognize, or be able to ascertain using no more than routine experimentation, equivalents to the specific embodiments of the teachings described herein. Such equivalents are intended to be encompassed by the following claims.

1. A high throughput method for detecting an amount of G6PDH in a biological sample, the method comprising:
reacting a biological sample with G6P and NADP in the presence of a surfactant and a buffer under suitable conditions to form 6-phosphogluconic acid;
quenching the biological sample to form a quenched sample; and
detecting by mass spectrometry a presence or amount of 6-phosphogluconic acid in the quenched sample,
wherein the amount of 6-phosphogluconic acid in the quenched sample is related to the amount of G6PDH in the biological sample.

2. The method of claim 1, wherein the quenching is accomplished using an enzyme denaturant.

3. (canceled)

4. The method of claim 1, wherein the mass spectrometry is accomplished using a mass spectrometer equipped with a thermally assisted electrospray ionization probe.

5. The method of claim 1, wherein the mass spectrometry is accomplished using a mass spectrometer equipped with a reversed phase liquid chromatography column.

6. The method of claim 1, wherein the detecting, occurs in less than about 5 minutes.

7. (canceled)

8. The method of claim 1, wherein the method allows for detection of about 0.01 milliUnits of G6PDH or less.

9. The method of claim 1, wherein the detecting is not substantially affected by fluctuations in temperature.

10. A method for detecting G6PDH deficiency in a subject, the method comprising
determining by mass spectrometry an amount of G6PDH in a biological sample from a subject; and
comparing the amount of G6PDH in the biological sample to an appropriate control, wherein G6PDH deficiency is detected when the amount of G6PDH in the sample is less than the appropriate control.

11. (canceled)

12. (canceled)

13. The method of claim 10, wherein an amount of G6PDH in the sample which is 10% less than the control indicates a G6PDH deficiency in the subject.

14. The method of claim 10, wherein an amount of G6PDH in the sample which is 25% less than the control indicates a G6PDH deficiency in the subject.

15. The method of claim 10, wherein an amount of G6PDH in the sample which is 50% less than the control indicates a G6PDH deficiency in the subject.

16. A method for diagnosing acute hemolytic anemia in a subject, the method comprising
detecting G6PDH deficiency in a subject in accordance with the method of claim 10, wherein a G6PDH deficiency in the subject indicates acute hemolytic anemia in the subject.

17. The method of claim 16, wherein an amount of G6PDH in the sample which is 25% less than the control indicates acute hemolytic anemia in the subject.

18. A method for diagnosing pre-eclampsia in as subject, the method comprising
detecting G6PDH deficiency in a subject in accordance with the method of claim 10, wherein a G6PDH deficiency in the subject indicates pre-eclampsia in the subject.

19. The method of claim 18, wherein an amount of G6PDH in the sample which is 25% less than the control indicates pre-eclampsia in the subject.

20. A prognostic method for increased mortality and/or morbidity resulting from pre-eclampsia or acute hemolytic anemia in a subject, comprising
detecting G6PDH deficiency in a subject in accordance with the method of claim 10, wherein an amount of the G6PDH in the biological sample which is 50% or less than the control indicates a prognosis of increased mortality and/or morbidity in the subject.

21. The method of claim 20, wherein the appropriate control is a control based upon G6PDH levels in a normal population.

22. The method of claim 20, further comprising:
reacting the biological sample with G6P and N ADP in the presence of surfactant and a buffer under suitable conditions to form 6-phosphogluconic acid;
quenching the biological sample to form a quenched sample; and
detecting by mass spectrometry a presence or amount of 6-phosphogluconic acid in the quenched sample,
wherein the amount of 6-phosphogluconic acid it the quenched sample is related to the amount of G6PDH in the biological sample.

23. (canceled)

24. A kit for detecting an amount of G6PDH in a biological sample, the kit comprising:
glucose-6-phosphate,
nicotinamide adenine dinucleotide phosphate,
optionally a buffer,
optionally a surfactant, and
instructions for preparing a reaction mixture that facilitates a reaction of nicotinamide adenine dinucleotide phosphate, glucose-6-phosphate and G6PDH.

25. The kit of claim 24, further comprising at least one of an enzyme denaturant, instructions for preparing a sample for analysis in a mass spectrometer, and a calibration curve which comprises a plot of [6-phosphogluconic acid/glucose-6-phosphate area] versus milliUnits of G6PDH.

26. (canceled)

27. (canceled)
